(19) United States
(12) Patent Application Publication
(10) Pub. No.: US 2022/0218374 A1
(43) Pub. Date: Jul. 14, 2022

(54) PROBIOTIC APPLICATIOR AND METHOD FOR USING SAME

(71) Applicant: New York University, New York, NY (US)

(72) Inventors: Deepak SAXENA, New York, NY (US); Xin LI, New York, NY (US)

(21) Appl. No.: 17/615,537

(22) PCT Filed: Jun. 1, 2020
(86) PCT No.: PCT/US2020/035620

§ 371 Date: Nov. 30, 2021

Related U.S. Application Data
(60) Provisional application No. 62/854,786, filed on May 30, 2019.

(51) Int. Cl. A61B 17/24; A61K 35/747; A61K 35/741; A61K 35/744; A61K 47/44; A61K 9/00; A61J 7/00

(52) U.S. Cl. CPC A61B 17/244; A61K 35/747; A61K 35/741; A61K 35/744; A61B 2017/00893; A61K 9/0053; A61K 9/0056; A61J 7/0053; A61K 47/44

(57) ABSTRACT

In some embodiments, the present disclosure provides a probiotic applicator, having a handle and a head affixed to the handle. The head is configured to scrape the surface of a tongue. The head includes a plurality of orifices for dispensing a fluid onto the surface of the tongue. The applicator includes a reservoir configured to contain a probiotic composition. The reservoir is in fluid communication with the plurality of orifices. An actuator is configured to allow probiotic composition to flow from the reservoir and through the plurality of orifices when actuated. In another aspect, the present disclosure may be embodied as a method for applying a probiotic composition to a tongue. The method includes scraping a surface of the tongue using a probiotic applicator having a scraping head; and applying the probiotic composition to the scraped surface of the tongue by dispensing through orifices of the probiotic applicator.

### CROSS-REFERENCE TO RELATED APPLICATIONS

This application claims priority to U.S. Provisional Application No. 62/854,786, filed on May 30, 2019, now pending, the disclosure of which is incorporated herein by reference.

### FIELD OF THE DISCLOSURE

The present disclosure relates to the use of probiotics, and more particularly to devices for applying probiotic compositions.

### BACKGROUND OF THE DISCLOSURE

Despite many improvements in oral hygiene, in the past, oral health continues to be a problem for many individuals. Conditions such as halitosis (i.e., bad breath) and periodontal disease are not only problematic to the physical person, but can also be a source of mental distress, causing anxiety and embarrassment.

### BRIEF SUMMARY OF THE DISCLOSURE

Probiotic applicators and methods for using such applicators are provided herein. Such applicators and methods may be used to apply probiotic compositions to the oral cavity for treatment of conditions such as, for example, periodontal disease and/or halitosis.

In some embodiments, the present disclosure provides a probiotic applicator, having a handle and a head affixed to the handle. The head is configured to scrape the surface of a tongue. The head includes a plurality of orifices for dispensing a fluid onto the surface of the tongue. The head may include a scrubber, and the plurality of orifices may be configured to dispense the probiotic composition into the scrubber. The scrubber may comprise bristles, knobs, and/or ridges, or other features to enhance cleaning action. The applicator includes a reservoir configured to contain a probiotic composition. The reservoir may be disposed within the handle and/or the head of the applicator. The reservoir is in fluid communication with the plurality of orifices. The reservoir may be configured to contain a probiotic composition under pressure An actuator is configured to allow probiotic composition to flow from the reservoir and through the plurality of orifices when actuated.

The applicator may include a probiotic composition contained within the reservoir. In an exemplary embodiment, the probiotic composition includes two or more of:

(a) Lactobacillus acidophilus,

(b) L. casei,

(c) L. reuteri,

(d) Akkermansia muciniphila,

(e) Ruminococcus bromii,

(f) Streptococcus salivarius, and

(g) one or more classes of phylum Verrucomicrobia and Firmicutes.

The probiotic composition may be present in a freeze-dried form. The probiotic composition may include 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli. The probiotic composition may further include coconut oil.

In another aspect, the present disclosure may be embodied as a method for applying a probiotic composition to a tongue. The method includes applying the probiotic composition to a surface of the tongue by dispensing through a plurality of orifices of a probiotic applicator. A surface of the tongue may be scraped using a scraping head of the probiotic applicator. Applying the probiotic composition may include depressing an actuator to commence flow of the probiotic composition from a reservoir to the orifices. Application of the probiotic composition may be done after the step of scraping the tongue and/or at the same time as the step of scraping the tongue.

The applied probiotic composition may include two or more of:

(a) Lactobacillus acidophilus,

(b) L. casei,

(c) L. reuteri,

(d) Akkermansia muciniphila,

(e) Ruminococcus bromii,

(f) Streptococcus salivarius, and

(g) one or more classes of phylum Verrucomicrobia and Firmicutes.

The probiotic composition may be present in a freeze-dried form. The probiotic composition may include 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli. The probiotic composition may further include coconut oil.

In another aspect, the present disclosure may be embodied as an oral rinse having two or more of:

(a) Lactobacillus acidophilus,

(b) L. casei,

(c) L. reuteri,

(d) Akkermansia muciniphila,

(e) Ruminococcus bromii,

(f) Streptococcus salivarius, and

(g) one or more classes of phylum Verrucomicrobia and Firmicutes.

The probiotic composition may be present in a freeze-dried form. The probiotic composition may include 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli. The probiotic composition may further include coconut oil.

In another aspect, the present disclosure may be embodied as a lozenge having two or more of:

(a) Lactobacillus acidophilus,

(b) L. casei,

(c) L. reuteri,

(d) Akkermansia muciniphila,

(e) Ruminococcus bromii,

(f) Streptococcus salivarius, and

(g) one or more classes of phylum Verrucomicrobia and Firmicutes.

The probiotic composition may be present in a freeze-dried form. The probiotic composition may include 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli. The probiotic composition may further include coconut oil.

### DESCRIPTION OF THE DRAWINGS

For a fuller understanding of the nature and objects of the disclosure, reference should be made to the following detailed description taken in conjunction with the accompanying drawings, in which:

FIG. 1A is a diagram of a probiotic applicator according to an embodiment of the present disclosure;

FIG. 1B is diagram of the probiotic applicator of FIG. 1A and showing certain internal components;

FIG. 2 is a diagram of a reservoir according to an embodiment of the present disclosure;

FIG. 3 is a diagram of a probiotic applicator according to another embodiment of the present disclosure;

FIG. 4 is a diagram of a probiotic applicator according to another embodiment of the present disclosure and showing certain internal components;

FIG. 5 is a diagram of a probiotic applicator according to another embodiment of the present disclosure;

FIG. 6 is a chart showing a method according to another embodiment of the present disclosure; and

FIG. 7 is a chart showing in vitro experimental results of an exemplary probiotic composition.

### DETAILED DESCRIPTION OF THE DISCLOSURE

Probiotic applicators and methods for using such applicators are provided herein. Such applicators and methods may be used to apply probiotic compositions to the oral cavity for treatment of conditions such as, for example, halitosis.

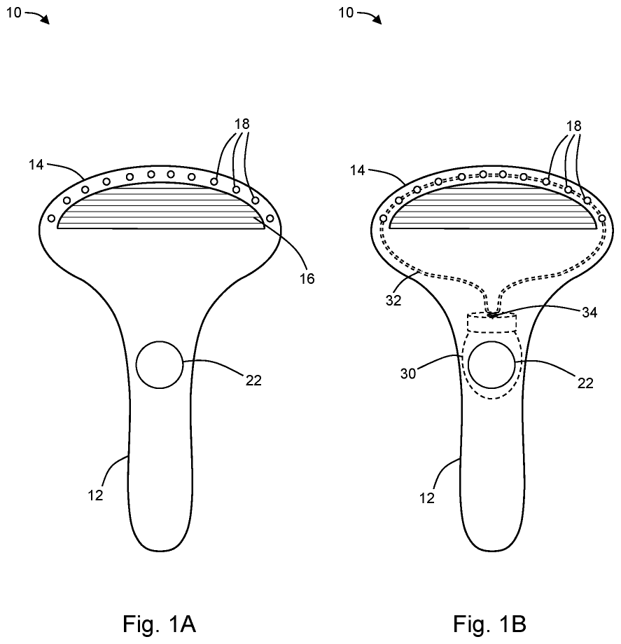

With reference to FIGS. 1A and 1B, the present disclosure may be embodied as a probiotic applicator 10. The probiotic applicator 10 includes a handle 12 and a head 14 affixed to the handle 12. The head 14 is configured to scrape the surface of a tongue. For example, the head 14 may be shaped so as to scrape a surface when the device is moved across the surface. In some embodiments, the head 14 includes a scrubber 16 for additional scraping (e.g., scrubbing) effectiveness. The scrubber 16 may include features such as, for example, bristles, knobs, and/or ridges, or any other such features. For example, the scrubber may have one or more ridges, each having an edge configured to cleaning the surface of the tongue. In some embodiments, the scrubber may be flexible so as to at least partially conform to the surface of the tongue. The scrubber may be made from any material suitable to the particular application, such as, polypropylene, polyethylene, nylon, rubbers (e.g., silicone, latex, etc.), metal, sponge (natural or artificial), etc., or combinations of one or more such materials.

The head 14 of the probiotic applicator 10 includes a plurality of orifices 18 for dispensing a fluid onto the surface of the tongue. The fluid may be, for example, a probiotic composition (as further described below), such as a probiotic mixture in a coconut oil base. In some embodiments, the applicator is configured such that when pulled across the tongue, the scrubber cleans (scrapes, wipes, etc.) the surface of the tongue before fluid is dispensed onto the tongue via the orifices (i.e., the scrubber leads the orifices). With reference to FIG. 3, in some embodiments, such as applicator 100, the orifices 118 may be positioned so as to dispense fluid into the scrubber 116 or through the scrubber 116.

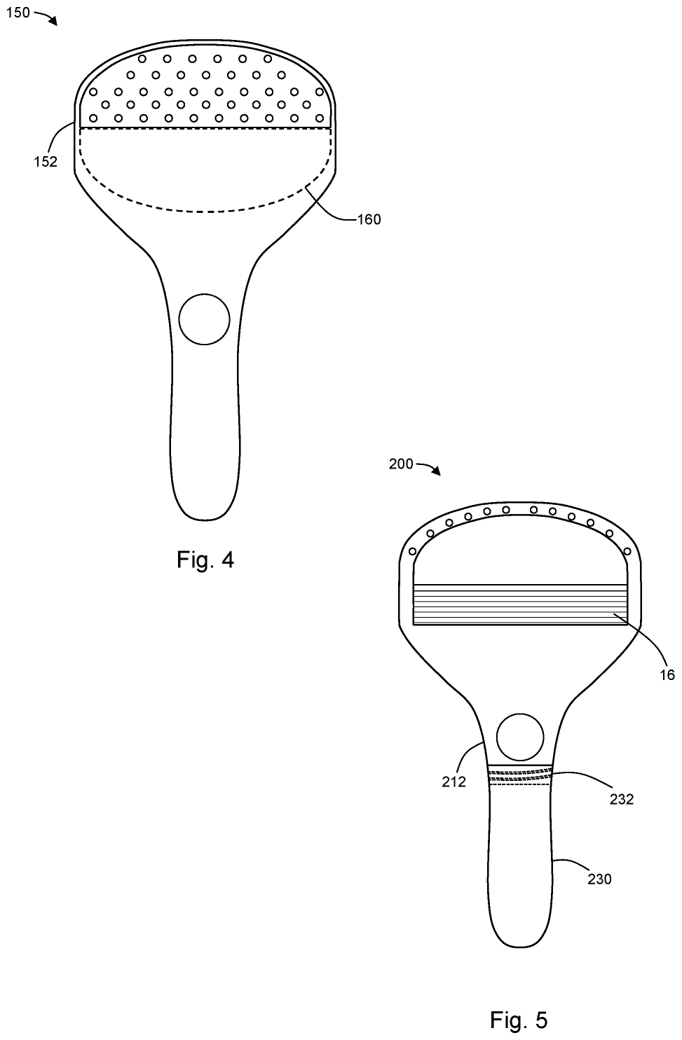

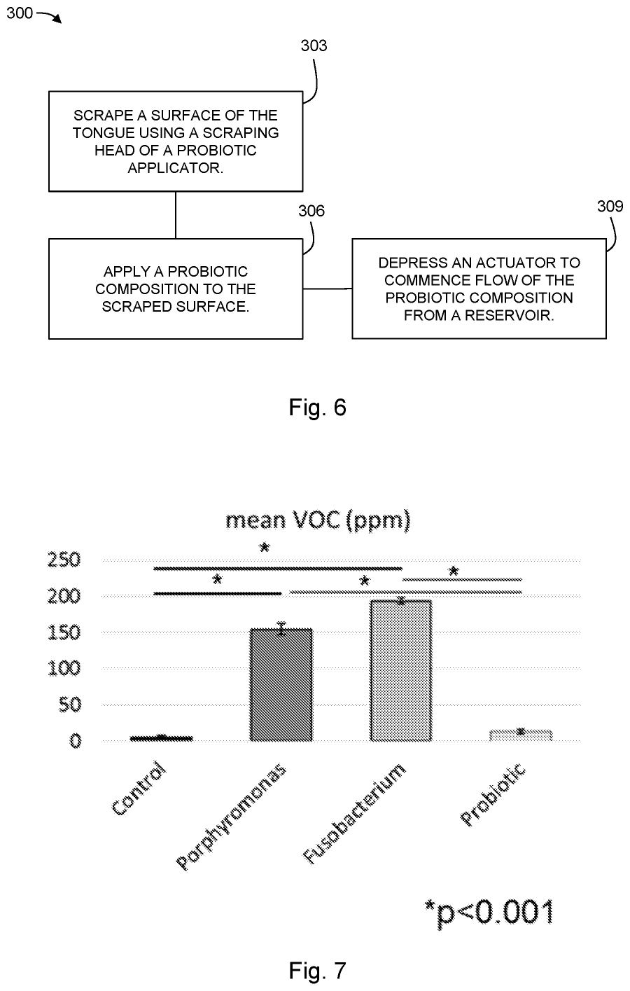

The applicator 10 has a reservoir 30 for containing the fluid. The reservoir 30 is in fluid communication with the plurality of orifices 18. In some embodiments (see, e.g., FIG. 4), the reservoir 160 is located in the head 152 of the applicator 150. In some embodiments, the reservoir 30 may be disposed in the handle 12. In various embodiments, the reservoir may be disposed partially in both the head and the handle or elsewhere. In some embodiments such as the applicator 200 depicted in FIG. 5, the reservoir 230 makes up a portion of the handle 212. Such a reservoir 230 may be removable from the applicator. For example, the reservoir 230 may be have screw threads 232 for attachment to corresponding screw threads of the handle 212. In some embodiments, the reservoir is a part of the head. In some embodiments, channels 32, such as, for example, tubes, are disposed through the head 14 to connect the reservoir to the orifices 18. Such channels may be, for example, formed (e.g., molded, machined, etc.) into the material of the head. In other embodiments, the channels comprise one or more separate tubes that are disposed in the head.

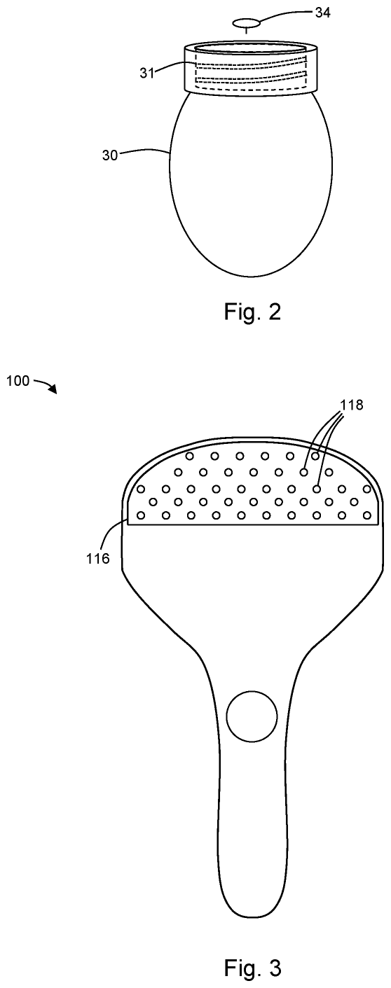

The reservoir 30 may contain a fluid comprising a probiotic composition. The reservoir 30 may be removable such that the applicator 10 can be refilled with probiotic composition by replacement of the removable reservoir. For example, with reference to FIG. 2, the reservoir 30 may include screw threads 31 for attachment to corresponding screw threads within the applicator, for example, within the handle. The handle 12 may include a needle 34, lance, blade, or other such device for piercing the reservoir in order to enable access to the probiotic composition. For example, the needle 34 may be positioned so as to pierce the reservoir 30 as the reservoir 30 is screwed into the handle 12. In some embodiments, the reservoir is not pierced until the actuator is actuated by a user. In other embodiments, there is no need for piercing the reservoir. For example, the reservoir may have a port with a cap or other cover which is removed before insertion in the applicator. In another example, the reservoir is not removable and there is no need for piercing or otherwise covering the reservoir which is filled and installed within the applicator. In some embodiments, the reservoir is a chamber formed (e.g., molded, machined, etc.) within the applicator.

Some embodiments of the probiotic applicator 10 include an actuator 22 to allow probiotic composition to selectively flow from the reservoir 30 to the plurality of orifices 18. The actuator 22 may be a button, a lever, a slide, a knob, or the like. In some embodiments, the reservoir 30 is configured to contain the probiotic composition under pressure. In this way, once the reservoir 30 is pierced, the probiotic composition will be biased to flow out through the plurality of orifices 18 without need for other motive force. In some embodiments, the actuator 22 is configured so as to stop the pressurized flow of probiotic composition unless/until the actuator 22 is actuated by a user. In some embodiments, the actuator 22 may be configured to allow a predetermined flow (e.g., flow for a pre-determined period of time, pre-determined volume, etc.) when actuated. In this way, the applicator 10 may be re-used by subsequent actuation of the actuator 22. In other embodiments, the actuator 22 is configured to allow a flow of probiotic composition for so long as the actuator 22 is actuated (e.g., begin flowing when a button is depressed and ending flow when the button is released). In some embodiments, once actuated, the probiotic composition will flow unimpeded (e.g., flow until substantially empty). In some embodiments, the reservoir is a pouch configured to release probiotic composition when compressed (e.g., squeezed). For example, the pouch may be manually squeezed by the hand of a user when using the applicator. In other embodiments, the pouch may be mechanically squeezed by, for example, a piston, a lever, etc. In some embodiments, the pouch may be squeezed by pneumatic or hydraulic pressure. For example, a pressurized gas cartridge may be used to compress the pouch and displace a fluid (e.g., probiotic composition) contained in the pouch.

In another aspect, the present disclosure may be embodied as a method 300 for applying a probiotic composition to a tongue. The method 300 may use a probiotic applicator such any of the embodiments described above. The method 300 includes applying 306 the probiotic composition to the scraped 303 surface of the tongue. For example, the probiotic composition may be dispensed through orifices of the probiotic applicator. An actuator may be actuated 309 to cause the flow of probiotic (e.g., a button may be depressed).

In some embodiments, the surface of the tongue is scraped 303 using a scraping head of the probiotic applicator. Scraping 303 may include a unidirectional motion (e.g., back to front) or a back-and-forth (e.g., scrubbing) motion over the surface of the tongue. In some embodiments, the probiotic composition is applied after the surface of the tongue is scraped. In some embodiments, the probiotic composition is applied at the same time as the tongue is scraped (e.g., during the scraping motion).

Probiotics refer to microorganisms that are considered to provide health benefits when administered to a subject. The probiotic compositions of the present disclosure include bacteria from one or more of Lactobacillus species, Akkermansia species, Ruminococcus species, Streptococcus species, Verrucomicrobia species, and Firmicutes species. In some embodiments, the probiotic composition comprises or consists essentially of two or more bacteria selected from: one or more Lactobacillus species, Akkermansia species, Ruminococcus species, Ochrobactrum species, Streptococcus species, Bifidobacterium species, Escherichia coli, Verrucomicrobia species, and Firmicutes species. In some embodiments, the probiotic composition comprises or consists essentially of lactobacillus and streptococcus species. The term “colony forming unit” or CFU may be used herein interchangeably with the number of bacteria. A CFU is a measure of viable bacterial cells.

Bacterial strains in an exemplary probiotic mixture (called “P00519”) suitable for halitosis (with exemplary quantities):

(i) Lactobacillus acidophilus,

(ii) L. casei,

(iii) L. reuteri,

(iv) Akkermansia muciniphila,

(v) Ruminococcus bromii,

(vi) Streptococcus salivarius, and

(ix) 3 strains belonging to phylum Verrucomicrobia and Firmicutes.

In some embodiments, the probiotic formulation may be represented as comprising bacteria comprising, consisting essentially of or consisting of:

(1) Lactobacillus acidophilus,

(2) L. casei,

(3) L. reuteri,

(4) Akkermansia muciniphila,

(5) Ruminococcus bromii,

(6) Streptococcus salivarius

(7) Verricomicrobia Verrucomicrobiae, and

(8) Firmicutes Bacilli

The bacteria may be isolated from animal or human fecal samples or may be obtained from a commercial source, such as American Tissue Type Collection (ATCC). To prepare the probiotic each bacteria type may be grown separately (such as by inoculation in appropriate broth, etc.) Following growth, bacteria may be isolated from the culture media, and bacteria may be lyophilized separately or may be combined in the desired amounts for a probiotic use.

In some embodiments, the disclosure provides a probiotic composition comprising, consisting essentially of, or consisting of the following bacteria: Lactobacillus acidophilus; L. casei, L. reuteri; Akkermansia muciniphila, Ruminococcus bromii; and strains of phylum Verrucomicrobia and phylum Firmicutes, and optionally, may contain one or more of Streptococcus thermophiles, Bifidobacterium breve, Escherichia coli Nissle 1917, Bifidobacterium lactis, and Streptococcus salivarius. The CFUs in the probiotics are provided as per dose.

The amount of bacteria (individual type or all types) per dose may be 100 million to 1 billion bacterial cells (i.e., CFUs) and all values and ranges therebetween. In some embodiments, a dose may have more than 1 billion bacteria (individual type or all types). A dose may be a tablet, capsule, or a specified amount of the formulation in any form. In various embodiments, the bacteria (individual type or all types) per dose may be 100, 200, 300, 400, 500, 600, 700, 800, 900 million or 1 billion, 2 billion, 3 billion, etc.

The composition can be formulated for oral administration. The present oral compositions may be in the form of a chewable formulation, a dissolving or dissolved formulation, an encapsulated/coated formulation, multi-layered lozenges (to separate active ingredients and/or active ingredients and excipients), a slow release/timed release formulation, or other forms suitable for oral delivery known in the art. It may be in the form of a tablet, lozenges, pill, capsule, drops, paste or the like. The formulations may also be present as encapsulated or incorporated into micelles, liposomes, cyclodextrins, polymers and the like.

The probiotic formulations, including pediatric formulations, may be flavored (e.g., fruit flavored, such as cherry, strawberry, blueberry etc.) and may be in a variety of shapes or colors.

In an aspect, the present disclosure provides pharmaceutically acceptable compositions which comprise a therapeutically-effective amount of one or more of the bacterial species as described above, the cell therapies as described above, and/or the cytokines as discussed above, formulated together with one or more pharmaceutically acceptable excipients. In an aspect, the present disclosure provides pharmaceutically acceptable compositions which comprise a therapeutically-effective amount of one or more of the bacteria species as described above, formulated together with one or more pharmaceutically acceptable excipients and other therapeutically effective medications known in the art allowing for but not limited to combination therapies to improve overall efficacy of each individual therapeutic or to limit the concentration of either therapeutic to avoid side effects and maintain efficacy. The active ingredient and excipient(s) may be formulated into compositions and dosage forms according to methods known in the art. The pharmaceutical compositions of the present invention may be specially formulated for administration in solid or liquid form, including those adapted for the following: oral administration, for example, tablets, capsules, powders, granules, and aqueous or non-aqueous solutions or suspensions, drenches, or syrups, frozen or freeze-dried forms. The probiotic compositions may be present in a lyophilized form (i.e., freeze-dried form). The bacteria may be lyophilized individually or the entire probiotic composition may be lyophilized.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Within the meaning of the disclosure, “treatment” also includes relapse, or prophylaxis as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

The phrase “pharmaceutically acceptable” is employed herein to refer to those compounds, materials, compositions, and/or dosage forms which are suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH-buffered solutions; and other non-toxic compatible substances employed in pharmaceutical formulations. If desired, certain sweetening and/or flavoring and/or coloring agents may be added. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

Excipients are added to the composition for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminum silicate, maltodextrin, methylcellulose, polymethacrylates, povidone (e.g., KOLLIDON®, PLASDONE®), pregelatinized starch, sodium alginate and starch.

In liquid pharmaceutical compositions of the present invention, the bacterial species and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present disclosure may also contain a viscosity enhancing agent to improve the mouth feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum. A liquid composition may also contain a buffer such as gluconic acid, lactic acid, citric acid or acetic acid, sodium gluconate, sodium lactate, sodium citrate or sodium acetate.

Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid. Preservatives and chelating agents such as alcohol, sodium benzoate, butylated hydroxy toluene, butylated hydroxyanisole and ethylenediamine tetraacetic acid may be added at levels safe for ingestion to improve storage stability.

The present compositions can be used to treat oral health conditions including halitosis. The compositions can also be used to improve immune response, oral hygiene, including bone density and treatment of bad breath. Halitosis is also a characteristic symptom of periodontal disease, and is caused by the production of volatile sulphur compounds (VSCs), such as hydrogen sulphide (H₂S) and methyl mercaptan, by sulfate-reducing bacteria. The major cultivatable periodontal opportunistic pathogens, Porphyromonas gingivalis (Pg), Fusobacterium nucleatum (Fn) and Tannerella forsythia (Tf), are reported to have produced H₂S in an in vitro system as measured by gas chromatography. These pathogens colonize the surface of tongue play significant role in H₂S production. In 80 to 90 percent halitosis cases involve bacteria from the oral cavity. Here we showed in vitro cell culture model and P00519 bacterial formulation to reduce H₂S production (FIG. 7).

The subject may be any animal, including human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Some specific embodiments of the present disclosure are provided below.

A lyophilized probiotic composition comprising, consisting essentially of or consisting of two or more or all of: Lactobacillus acidophilus (10⁸ CFUs); L. casei (10⁸ CFUs); L. reuteri (10⁸ CFUs); Akkermansia muciniphila (10⁷ CFUs); Ruminococcus bromii(10⁷ CFUs); Streptococcus salivarius (10⁹ CFUs); and strains of phylum Verrucomicrobia and Firmicutes (10⁶ CFUs).

In some embodiments, the present probiotic compositions may be used in the treatment of halitosis. For example, P00519 may be used directly in the oral cavity for the treatment of halitosis. The probiotic may be used by itself in the form of a rinse, paste, liquid, gel or chewable or other tablets, or may be incorporated into toothpastes, other rinses (such as dental or oral mouthwashes), oral or dental appliances, or any other device that may come in contact with the oral cavity.

The probiotic composition can be formulated for administration using the applicator described above. For example, the composition may be formulated as a liquid that can be contained in the reservoir, flowed through the channels, and dispensed by way of the plurality of orifices. Some compositions have a coconut oil liquid in which the probiotic mixture is mixed. One or more other liquids may be used in addition to, or instead of, coconut oil. The probiotic formulations, including pediatric formulations, may be flavored (e.g., fruit flavored, such as cherry, strawberry, blueberry, etc.) and/or may be in a variety of colors.

The present applicator and method can be used with probiotic compositions to treat oral conditions including halitosis. The compositions can also be used to improve oral hygiene and treatment of bad breath. Halitosis is also a characteristic symptom of periodontal disease, and is caused by the production of volatile sulphur compounds (VSCs), such as hydrogen sulphide (H₂S) and methyl mercaptan, by sulfate-reducing bacteria. The major cultivatable periodontal opportunistic pathogens, Porphyromonas gingivalis (Pg), Fusobacterium nucleatum (Fn) and Tannerella forsythia (Tf), are reported to have produced H₂S in an in vitro system as measured by gas chromatography. These pathogens can colonize the surface of tongue and play a significant role in H₂S production. In 80 to 90 percent of halitosis cases, bacteria from the oral cavity are involved. FIG. 7 shows results of an in vitro cell culture model and P00519 bacterial formulation to reduce H₂S production (further described below).

Halitosis, bad breath or oral malodor are all synonyms for the same pathology. Halitosis has a large social and economic impact. For the majority of patients suffering from bad breath, it causes embarrassment and affects their social communication and life. Moreover, halitosis can be indicative of underlying diseases. Halitosis is also a characteristic symptom of periodontal disease, and is caused by the production of volatile Sulphur compounds (VSCs), such as hydrogen sulphide (H₂S) and methyl mercaptan, by sulfate-reducing bacteria. Importantly, the major cultivatable periodontal opportunistic pathogens, Porphyromonas gingivalis (Pg), Fusobacterium nucleatum (Fn) and Tannerella forsythia (Tf), are reported to have produced H₂S in an in vitro system as measured by gas chromatography. These pathogens colonize the surface of tongue play significant role in H₂S production. In 80 to 90 percent halitosis cases involve bacteria from the oral cavity. Here we used in vitro cell culture model and P00519 bacterial formulation to determine whether cocktail of certain bacteria can reduce H₂S production.

Measurement of Hydrogen Sulfide from Bacteria.

Probiotic composition P00519 was tested using two major bacteria Porphyromonas spp and Fusobacterium spp involved in halitosis and periodontal diseases. Bacteria were cultured in broth medium until they reached late log growth phase, and the concentration of all strains was then adjusted to 10⁸-10⁹ cell/ml. Subsequently, the bacterial suspension was used for detecting H₂S production in bacterial biofilm culture and in presence of oral epithelial cells.

Calorimetric method: The bismuth sulfide method was modified by using a 5 mM concentration of bismuth(III)chloride. Bacteria were diluted in peptone solution to 10⁹ cells/mL. Aliquots (100 ml) of the bacterial suspension were mixed with an equal amount of newly prepared bismuth solution (0.4 M triethanolamineHCl, pH 8.0; 10 mM bismuth(III)chloride; 20 mM pyridoxal 5-phosphate monohydrate, 20 mM EDTA and 40 mM L-cysteine) in microtiter plates. H₂S production was monitored by detecting black BS is precipitated. Intensity of black precipitate was visually scaled, from no color production (0) to maximum black color production after 24 hours.

H₂S production in epithelial cell and bacterial co-culture model: Human oral epithelial origin cell line OKF6 was used in this study. The cells were cultured in Keratinocyte-Serum Free Medium supplemented with 50 μg/ml bovine pituitary extract, 5 ng/ml epidermal growth factor in humidified atmosphere of 5% CO₂ at 37° C. The bacterial species tested for H₂S-producing capacity were Porphyromonas spp, Fusobacterium spp, and probiotic cocktail (P00519). The species were grown on appropriate agar plates under optimal conditions. Desired concentration of theses bacteria or P00519 (10⁸ cfu/ml) was added to 2 M epithelial cells in 25 ml Corning Primaria Tissue Culture Flasks and incubated for 24 hours as mentioned before. Handheld Hydrogen Sulfide (H₂S) Gas Detector with range from 0 to 500 ppm was used for H₂S production.

### Results:

In calorimetric method bacterial hydrogen sulfide (H₂S) production from cysteine measured with colorimetric methods in microtiter plate format, recorded as black bismuth sulfide precipitation formation. The most rapid H₂S production was seen for Porphyromonas spp and Fusobacterium spp. reaching the maximum color production then the probiotic group and controls.

Further, we used H₂S as marker of production of volatile Sulphur compounds in association with oral epithelial cells. The results indicated that when cell were co-cultured with Porphyromonas spp and Fusobacterium spp there was higher production of H₂S whereas when the cells were co-cultured with probiotic cocktail (P00519) and without bacteria there was significantly less production of H₂S (FIG. 7). This results indicated that Porphyromonas and Fusobacterium can utilize the components of media and epithelial cells to produce H₂S whereas use of the exemplary probiotic cocktail does not produce volatile sulphur compounds. These data indicate the present probiotic compositions can be used for the prevention and/or treatment of bad breath.

In another aspect, the present disclosure provides pharmaceutically acceptable compositions which comprise a therapeutically-effective amount of one or more of the bacterial species as described above, the cell therapies as described above, and/or the cytokines as discussed above, formulated together with one or more pharmaceutically acceptable excipients. In another aspect, the present disclosure provides pharmaceutically acceptable compositions which comprise a therapeutically-effective amount of one or more of the bacteria species as described above, formulated together with one or more pharmaceutically acceptable excipients and other therapeutically effective medications known in the art allowing for but not limited to combination therapies to improve overall efficacy of each individual therapeutic or to limit the concentration of either therapeutic to avoid side effects and maintain efficacy. The active ingredient and excipient(s) may be formulated into compositions and dosage forms according to methods known in the art.

The phrase “pharmaceutically acceptable” is used herein to refer to those compounds, materials, compositions, and/or dosage forms which are suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid) or solvent, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g., in “Remington's Pharmaceutical Sciences,” The Science and Practice of Pharmacy, 19th Ed. Mack Publishing Company, Easton, Pa., (1995).

Excipients are added to the composition for a variety of purposes. For example, diluents increase the bulk of a pharmaceutical composition, and may make a pharmaceutical dosage form containing the composition easier for the patient and caregiver to handle.

In liquid pharmaceutical compositions of the present disclosure, the bacterial species and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present disclosure include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present disclosure may also contain a viscosity enhancing agent to improve the mouth feel of the product and/or coat the lining of the gastrointestinal tract.

Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum. A liquid composition may also contain a buffer such as gluconic acid, lactic acid, citric acid or acetic acid, sodium gluconate, sodium lactate, sodium citrate or sodium acetate.

Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Common flavoring agents and flavor enhancers for pharmaceutical products that may be included in the composition of the present disclosure include maltol, vanillin, ethyl vanillin, menthol, citric acid, fumaric acid, ethyl maltol and tartaric acid. Preservatives and chelating agents such as alcohol, sodium benzoate, butylated hydroxy toluene, butylated hydroxyanisole and ethylenediamine tetraacetic acid may be added at levels safe for ingestion to improve storage stability.

Although the present disclosure has been described with respect to one or more particular embodiments, it will be understood that other embodiments of the present disclosure may be made without departing from the spirit and scope of the present disclosure.

We claim:

1. A probiotic applicator, comprising:
a handle;
a head affixed to the handle and configured to scrape the surface of a tongue, the head further comprising a plurality of orifices for dispensing a fluid onto the surface of the tongue;
a probiotic reservoir configured to contain a probiotic composition, wherein the probiotic reservoir is in fluid communication with the plurality of orifices; and
an actuator configured to allow probiotic composition to flow from the reservoir and through the plurality of orifices when actuated.

2. The probiotic applicator of claim 1, wherein the reservoir is disposed within the handle.

3. The probiotic applicator of claim 1, wherein the reservoir is disposed within the head.

4. The probiotic applicator of claim 1, wherein the head comprises a scrubber.

5. The probiotic applicator of claim 4, wherein the plurality of orifices are configured to dispense the probiotic composition into the scrubber.

6. The probiotic applicator of claim 4, wherein the scrubber comprises bristles, knobs, and/or ridges.

7. The probiotic applicator of claim 1, wherein the reservoir is configured to contain the probiotic composition under pressure.

8. The probiotic applicator of claim 1, further comprising a probiotic composition contained within the reservoir.

9. The probiotic applicator of claim 8, wherein the probiotic composition comprises two or more of:
(a) Lactobacillus acidophilus,
(b) L. casei,
(c) L. reuteri,
(d) Akkermansia muciniphila,
(e) Ruminococcus bromii,
(f) Streptococcus salivarius, and
(g) one or more classes of phylum Verrucomicrobia and Firmicutes.

10. The probiotic applicator of claim 9, wherein the probiotic composition is present in a freeze-dried form.

11. The probiotic applicator of claim 10, wherein the probiotic composition comprises 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli.

12. The probiotic applicator of any one of claims 8-11, wherein the probiotic composition further comprises coconut oil.

13. The probiotic applicator of claim 1, wherein the reservoir is removable.

14. The probiotic applicator of claim 1, wherein the actuator is configured to cause the reservoir to be pierced to cause a flow of probiotic composition.

15. The probiotic applicator of claim 1, wherein the reservoir is a pouch configured to release probiotic composition when squeezed.

16. The probiotic applicator of claim 1, wherein the actuator is a button, a lever, a slide, or a knob.

17. A method for applying a probiotic composition to a tongue, comprising:
applying the probiotic composition to a surface of the tongue by dispensing through a plurality of orifices of a probiotic applicator.

18. The method of claim 17, further comprising depressing an actuator to commence flow of the probiotic composition from a reservoir to the plurality of orifices.

19. The method of claim 17, further comprising scraping a surface of the tongue using a scraping head of the probiotic applicator.

20. The method of claim 19, wherein the step of applying probiotic composition is done after the step of scraping the tongue.

21. The method of claim 19, wherein the step of applying probiotic composition is done at the same time as the step of scraping the tongue.

22. The method of claim 17, wherein the probiotic composition comprises two or more of:
(a) Lactobacillus acidophilus,
(b) L. casei,
(c) L. reuteri,
(d) Akkermansia muciniphila,
(e) Ruminococcus bromii,
(f) Streptococcus salivarius, and
(g) one or more classes of phylum Verrucomicrobia and Firmicutes.

23. The method of claim 22, wherein the probiotic composition is present in a freeze-dried form.

24. The method of claim 23, wherein the probiotic composition comprises 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli.

25. The method of any one of claims 21-24, wherein the probiotic composition further comprises coconut oil.

26. An oral rinse comprising a probiotic composition comprising two or more of:
(a) Lactobacillus acidophilus,
(b) L. casei,
(c) L. reuteri,
(d) Akkermansia muciniphila,
(e) Ruminococcus bromii,
(f) Streptococcus salivarius, and
(g) one or more classes of phylum Verrucomicrobia and Firmicutes.

27. The oral rinse of claim 22, wherein the probiotic composition is present in a freeze-dried form.

28. The oral rinse of claim 27, wherein the probiotic composition comprises 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli.

29. A lozenge comprising a probiotic composition comprising two or more of:
(a) Lactobacillus acidophilus,
(b) L. casei,
(c) L. reuteri,
(d) Akkermansia muciniphila,
(e) Ruminococcus bromii,
(f) Streptococcus salivarius, and
(g) one or more classes of phylum Verrucomicrobia and Firmicutes.

30. The lozenge of claim 29, wherein the probiotic composition is present in a freeze-dried form.

31. The lozenge of claim 30, wherein the composition comprises 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli.
